(19) United States
(12) Patent Application Publication
(10) Pub. No.: US 2007/0021839 A1
(43) Pub. Date: Jan. 25, 2007

(54) Trapezium prosthesis and method

(76) Inventor: William Lowe, Fort Worth, TX (US)

Correspondence Address: WHITAKER, CHALK, SWINDLE & SAWYER, LLP, 3500 CITY CENTER TOWER II, 301 COMMERCE STREET, FORT WORTH, TX, 76102-4186, US

(21) Appl. No.: 11/186,569

(22) Filed: Jul. 21, 2005

(51) Int. Cl. A61F 2/42

(52) U.S. Cl. 623/21.11; 623/21.15; 623/13.14

(57) ABSTRACT

A trapezium prosthesis is provided for replacement of a diseased trapezium of a hand. Also shown is a surgical technique for placement of the prosthesis. Rather than utilizing hemiarthoplasty or total joint replacement, the trapezium is replaced with the prosthesis made of a material other than silicon. The prosthesis has specially prepared surfaces to encourage the growth and attachment of surrounding tissue.

### BACKGROUND OF THE INVENTION

1. Field of the Invention

The present invention relates generally to the field of surgical prosthetic devices used in orthopedic medicine and, more specifically, to a trapezium replacement prosthesis which can be used for the treatment of thumb basil joint arthritis or other conditions and to a method of its use.

2. Description of the Prior Art

Conditions such as osteoarthritis, cancer, trauma and infection may cause degeneration of the articular surfaces between the trapezium and the first metacarpal in a person's hand causing the patient discomfort and sometimes severe pain during use and movement of the thumb.

Various total replacement prosthesis have been proposed for bones in the human wrist other than the trapezium. For example, U.S. Pat. No. 4,936,860 to Swanson, issued Jun. 26, 1990, discloses a metallic total scaphoid replacement implant. U.S. Pat. Nos. 4,955,915 and 4,969,908, also issued to Swanson on Sep. 11, 1990 and Nov. 13, 1990, respectively, disclose total lunate replacement implants.

Excision of the trapezium without prosthetic replacement has also been carried out in the past for treatment of osteoarthritis and other conditions. The simple trapeziectomy has gained widespread acceptance in the past, but is not ideal due to reports of postoperative weakness. More recently, ligament reconstruction techniques have been the favored surgical procedure for the treatment of trapezial arthritis with various interposition material incorporated into the trapezial void. See, for example, “Trapezium Excision and Ligament Reconstruction With Abductor Pollicis Longus for Basal Arthritis of the Thumb”, Leger and Lavalle, Techniques In Hand and Upper Extremity Surgery”, 7(3); 98-101, 2003. See also, “Ligament Reconstruction Tendon Interposition Arthroplasty For Basal Joint Arthritis”, Tomaino, Hand Clinics, Department of Orthopaedic Surgery, University of Pittsburgh Mdical Center, Pittsburgh, Pa., Vol. 17, Number 2, May 2001. The necessity for such an extensive procedure that requires tendon harvest and rerouting has come into question, however. See, for example, “Hematoma and Distraction Arthroplasty For Basal Thumb Osteoarthritis”, by Kuhns and Meals, University of California, Los Angeles, School of Medicine, Techniques in Hand and Upper Extremity Surgery, 8(1); 2-6, 2004.

As an alternative to total carpal bone replacement, it is well known in the art to install a prosthetic implant. For example, the Silastic RTM Trapezial Implant HP™, manufactured by Dow Corning Wright Corp., 5667 Airline Road, Arlington, Tenn. 38002, is made from medical grade silicone rubber elastomer for use as an interpositional spacer between the trapezium and the first metacarpal joint of the thumb. The implant is provided with a short cylindrical stem extending from its distal surface which fits into a cavity prepared in the metacarpal. The silastic implants rely upon the stem in the metacarpal and the soft-tissue (ligaments and scar tissue) envelope for stabilization. Silicone implant arthroplasty has fallen out of favor in some instances, however, due to problems of subluxation and reaction to silicone wear debris. In some cases of implant arthroplasty, the implant loosens with time causing patient discomfort and, on occasion, repeat surgery.

A need exists, therefore, for continued improvements in the surgical treatment of osteoarthritis and other related conditions which affect the thumb basil joint.

A need exists for a surgical treatment for diseases of the thumb basil joint which overcomes problems associated with loosening of prior art implants used in arthroplastic reconstruction of the human hand and its joints.

A need also exists for an improved prosthesis which can be used to replace an excised trapezium in the human hand which overcomes many of the difficulties of the prior art techniques.

### SUMMARY OF THE INVENTION

The present invention is a prosthetic device for replacing a trapezium in a human hand, for example, in the treatment of thumb basil joint arthritis. The prosthetic device is a specially formed synthetic body sized and shaped to resemble the removed trapezium. The body has a first articulating surface arranged for engagement with a proximal end of a first metacarpal in the hand through a thumb basilarjoint. The device has an oppositely, second articulating surface arranged for engagement with a distal end of a scaphoid bone of the hand through a scapho trapezio trapezoidjoint. The body has a third articulating surface which is arranged for engagement with a trapezoid bone and the second metacarpal bone of the hand. The body also has at least one additional surface which has been formed to promote attachment and adhesion of surrounding ligaments or other tissues in the hand after placement of the prosthetic device.

Preferably, the surface which has been formed to promote attachment and adhesion of surrounding ligaments and tissue in the hand is either a porous surface or a specially roughened surface which is provided on a side of the body other than the articulating surfaces. A plurality of such surfaces are preferably provided. Alternatively or in addition to the porous or specially roughened surface, the body may be provided with at least one hole which passes wholly or partly through the body to facilitate attachment of surrounding ligaments or tissues of the hand. The synthetic body may be formed of a variety of materials including ceramics, titanium, stainless steel, synthetic diamond and other medical grade super alloys based on nickel and/or cobalt, and the like.

In the surgical method of the invention, the trapezium is first excised from the hand of a human. The excised trapezium is then replaced with a synthetic body as previously described which is sized and shaped to resemble the removed trapezium. Surrounding ligaments or other tissues in the hand are then attached to the specially prepared surfaces of the prosthetic device.

Additional objects, features and advantages will be apparent in the written description which follows.

### BRIEF DESCRIPTION OF THE DRAWINGS

FIG. 1 is a fragmentary, posterior or dorsal view of the wrist joint of the left hand showing the distal and proximal carpal rows.

FIG. 2 is a view similar to FIG. 1 but showing the excised trapezium region in preparation for placement of a prosthesis in accordance with the teaching of the invention.

FIG. 3 is a fragmentary, anterior view of the wrist of a human showing portions of the distal and proximal carpal rows and the extrinsic ligaments of the wrist.

FIGS. 4 and 5 are views of the respective components of prior art implants used in treatment of thumb basil joint arthritis.

FIGS. 6-8 are simplified views of a typical prior art surgical procedure in which the components of FIGS. 4 and 5 are implanted in a human hand.

FIG. 9 is an isolated view of the trapezium prosthesis of the invention.

FIGS. 10-12 are simplified, schematic views of the specially prepared surfaces of the prosthesis which are used to attach the surrounding ligaments in the hand.

FIGS. 13-15 are simplified views of the surgical procedure of the invention in which the prosthesis is used to replace a diseased trapezium in a human hand.

### DETAILED DESCRIPTION OF THE INVENTION

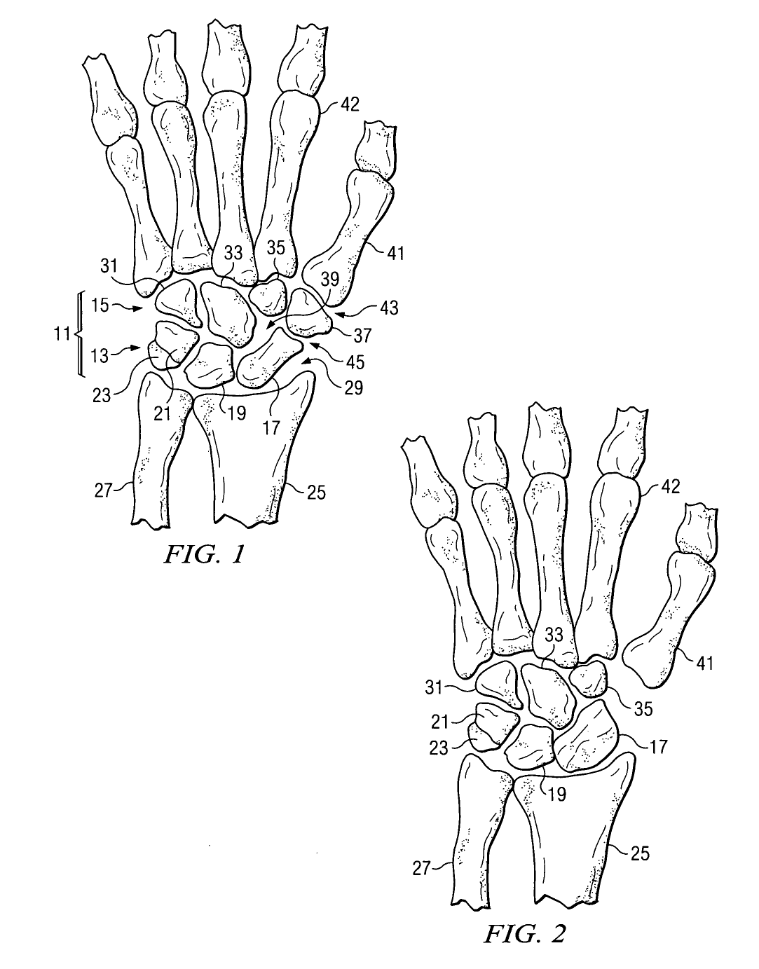

Turning now to FIGS. 1 and 2, there is shown a fragmentary posterior or dorsal view of the wrist joint of a left hand. Wrist joint 11 includes a proximal carpal row 13 and a distal carpal row 15. Proximal carpal row 13 includes a scaphoid bone 17, a lunate bone 19, a triquetrum bone 21 and a pisiform bone 23. The proximal row 13 is adjacent the radius 25 and the ulna 27. A joint 29 extends along the proximal carpal row of the wrist between the distal radius. Joint 29 is referred to as the radiocarpal joint. Distal carpal row 15 includes the hamate bone 31, the capitate bone 33, the trapezoid bone 35 and the trapezium bone 37. The midcarpaljoint 39 extends between the distal and proximal carpal rows. The trapezium bone 37 articulates proximally about the scaphoid bone 17, distally about the thumb metacarpal 41 and medially about the second metacarpal 42 and trapezoid bone 35. The trapezium bone 37 is related to the thumb metacarpal distally through the thumb basilar joint 43 and proximally to the scaphoid through the scapho trapezio trapezoid joint 45.

FIG. 3 is intended to be a simplified representation of the various surrounding ligaments of the principal bones of the hand discussed above. As shown in FIG. 3, the carpal bones are held together by short, interosseous ligaments. The ulnar collateral and radial collateral ligaments provide lateral support for the wrist. Palmar radiocarpal and dorsal radiocarpal ligaments maintain support of the carpal area. The fibers of the palmar radiocarpal ligament extend distally and obliquely from the radius, the triangular fibrocartilage and styloid process of the ulna. These ligaments define a symmetrical pattern due to insertions into the scaphoid, lunate, triquetrum and capitate bones.

The above discussion is intended to set the environment of the present invention which involves surgical removal of the trapezium bone 37 and its total replacement with a synthetic prosthesis. The anatomical features discussed above will be well familiar to those skilled in the field of orthopedic medicine.

In order to best explain the trapezium prosthesis of the invention and the surgical method of its use, reference will first be had to FIGS. 6-8 which illustrate a prior art trapezium implant technique.

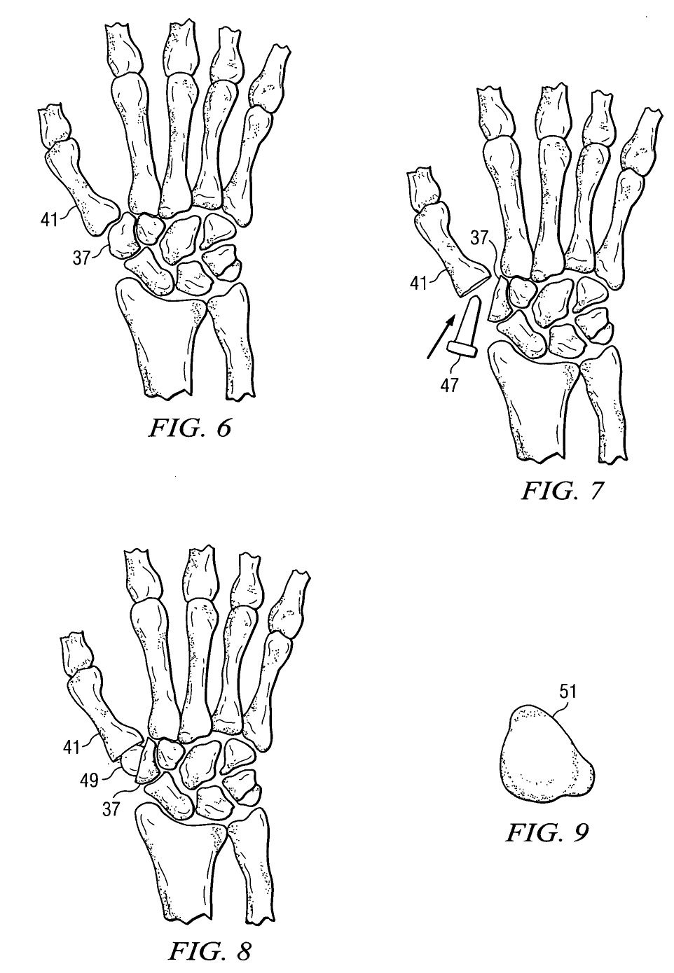

FIG. 6 shows the trapezium 37 prior to beginning the procedure. The technique shown in FIG. 7 is a silastic implant total joint procedure, since the removal of both of the joint surfaces is involved. The “hemiarthroplasty” illustrated in FIG. 8 is a similar procedure, but only half the joint (one of the articulating surfaces) will be replaced. Referring back to FIG. 7, the trapezium 37 is prepared as follows. The carpometacarpal joint of the thumb is exposed through a transverse or curved longitudinal incision. The superficial sensor branch of the radial nerve is identified and protected. The abductor pollices longus tendon is detached at its insertion and the extensor pollices brevis tendon is retracted. The joint capsule is opened and preserved. The marginal osteophytes at the base of the metacarpal 41 and trapezium 37 are removed with a rongeur. Distal traction of the thumb allows the joint surfaces to be visualized. The saddle of the trapezium 37 is then converted to a flat surface and the medial osteophyte is removed. Excision of this medial spur permits further debridement of the capsule and reduction of the subluxed metacarpal. FIG. 6 illustrates the distal articular portion or saddle resected from the trapezium 37. This saddle is excised to form a stable, transverse interface surface. The base of the thumb metacarpal 41 is similarly prepared. FIG. 7 illustrates the insertion of a silastic style implant into the base of the thumb metacarpal 41.

Another commercially available implant is the Ascension CMC™ style implant (available from Ascension Orthopedics, Inc., 8200 Cameron Road, Austin, Tex.) The CMC™ style implant is formed of a pyrocarbon material and has a unique head shape with a saddle-like articular surface that is shaped somewhat like a conventional bicycle seat. The device can be implanted into the medullary canal of the first metacarpal after removing the base of that bone using a “hemiarthoplasty” technique.

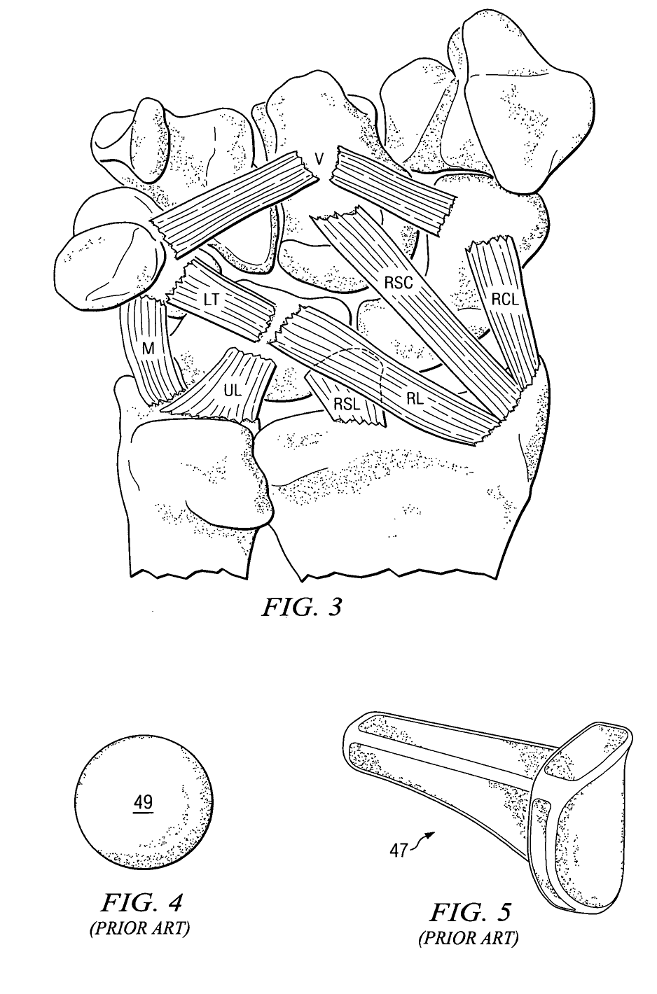

FIG. 8 illustrates an alternative BioSphere™ implant which is also commercially available and has been used in the prior art. As shown in FIG. 4, this implant utilizes a spherical body 49 which is fitted to the resected saddle region of the trapezium 37. The spherical or semi-spherical component 49 may be fabricated, for example, from an inelastic and abrasion resistant material, such as commercially pure titanium, other medical grade metals or alloys, or alumina, zirconia or silica ceramics. Because of the debris problems previously discussed, the prosthesis is preferably not constructed of silicon. Other known materials used in fabricating implants of the type under consideration include zirconium with a coating of zirconium oxide for improved abrasion resistance as well as zirconium alloys including titanium, niobium, hafnium and other metals known to form stable alloys with zirconium.

As discussed in the Background portion of the Specification, implants of the above type have, in many cases, proved to be problematical over time. Implants of whatever shape or design have a tendency to loosen with the passage of time. Applicant's surgical approach does not attempt to provide a new joint surface on an old bone, as in the above implant techniques. Rather, Applicant's improved trapezium prosthesis replaces the entire trapezium, while at the same time providing specially prepared surfaces to facilitate attachment of surrounding ligaments or other tissues.

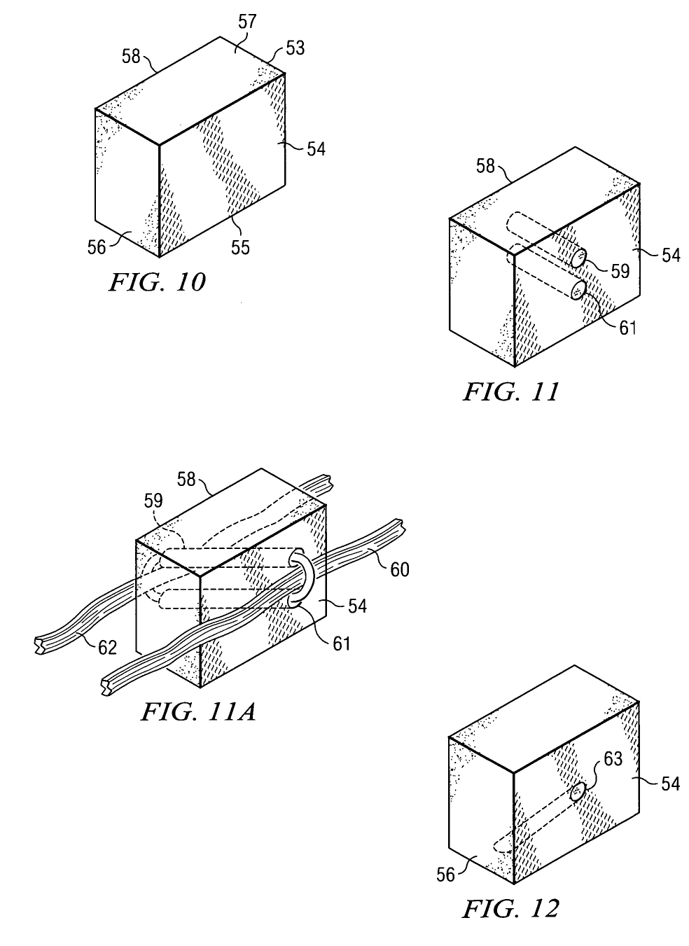

FIG. 9 illustrates the improved trapezium prosthesis of the invention, designated generally as 51. The prosthesis 51 is shaped and sized to replicate the original trapezium bone (37 in FIG. 1). The prosthesis will not necessarily be an exact duplicate of the excised bone, however. Of primary importance is the fact that the prosthesis adequately occupies the trapezial void and that it present adequate “attachment surfaces” for surrounding tissues. For example, with reference to FIGS. 10-12, the prosthesis can be analogized to the shape of a simple cube having sides 53, 54, 55, 56, 57 and 58.

In order to insure a proper fit, the prosthesis in accordance with the present invention is fabricated in proportionally increased sizes. Also, due to the mirror image differences between the right and left wrist, right and left mirror image prosthesis devices must be provided. Larger sized prostheses basically result from proportional size increases. The prosthesis will thus be provided in a range of sizes to accommodate variations in hand sizes of individuals but will otherwise be similar in appearance to the natural bone being excised. Suitable materials for the prosthesis include, for example, ceramics, titanium, stainless steel, synthetic diamond and medical grade super alloys based on nickel and/or cobalt-chrome as well as the other known materials mentioned with respect to the prior art discussion above.

Synthetic diamond is the most abrasion resistant material available and is one of the least toxic materials to the human body. Synthetic diamond offers potential advantages over many of the more traditional prosthetic materials, particularly in terms of wear resistance and a low coefficient of friction at the bearing surfaces. A suitable synthetic diamond material has recently been offered in the marketplace for total hip replacement by Exactech, Inc., of Gainesville, Fla.

As discussed above, the prosthesis 51 will be provided with specially prepared surfaces to facilitate the attachment of surrounding ligaments and tissues (joint capsule, muscle facia, etc.) in the hand. The body has a first articulating surface (57 in FIG. 10) arranged for engagement with a proximal end of the first metacarpal in the hand through the thumb basilar joint. The body of the prosthetic device has a second articulating surface 55 arranged for engagement with a distal end of the scaphoid bone of the hand through the scapho trapezio trapezoid joint. The body has a third articulating surface 53 which is arranged for engagement with the trapezoid bone 35 and the second metacarpal bone 41 of the hand. The remaining sides of the cube, e.g., sides 54, 56 and 58, present available attachment surfaces which can be prepared to promote attachment and adhesion of surrounding ligaments and other tissues in the hand after placement of the prosthetic device.

For example, the surface 54 which has been formed to promote attachment and adhesion of surrounding ligaments in the hand may be a roughened or porous surface which is provided on a side of the body other than the articulating surfaces. Additionally, as shown in FIG. 11, the body of the device may be provided with at least one and preferably a plurality of holes 59, 61 which pass part way or entirely through the body between the sides thereof to facilitate attachment of surrounding ligaments and tissues of the hand. For example, the holes 59, 61 maybe used to anchor sutures with material left hanging out to attach surrounding ligaments or tissues. The sutures then provide a means for holding the surrounding soft tissues to the specially prepared “in-growth” surfaces while the ligaments and scar tissue attaches to the prosthesis during the healing process. As shown in FIGS. 11 and 12, the attachment holes 59, 61 and 63 may be in parallel or non-parallel planes and pass between opposite sides of the body or may pass through adjacent sides of the body to facilitate attachment of surrounding ligaments and tissues of the hand.

FIG. 11A shows ligaments 60, 62 passing proximate the sides 54 and 58 of the cube, respectively. Sutures can be passed through the holes 59, 61 and can be tied down with a knot to “cinch” the ligaments down against the respective cube sides. This type contact secures the soft tissues (ligaments, capsule, facia, etc) to the three “in-growth” or attachment surfaces of the prosthesis body.

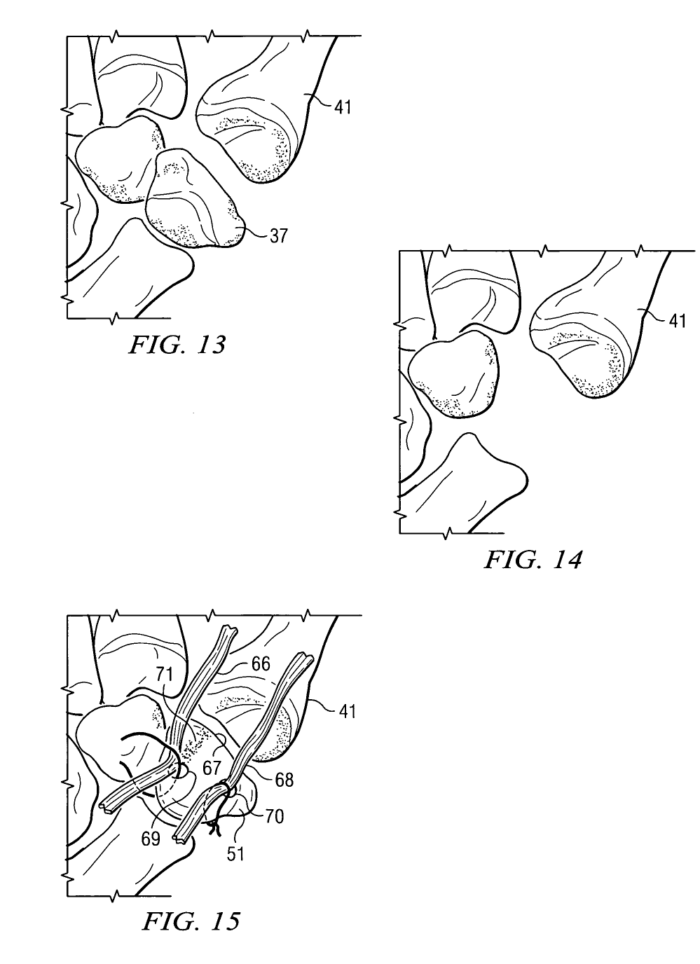

FIGS. 13-15 are intended to illustrate, in simplified fashion, the surgical procedure for excision of the trapezium, installation of the prosthesis of the invention and the reattachment of the surrounding ligaments or other tissues. The disection or exposure is generally the same as that described with respect to the prior art hemiarthroplasty, except that the entire trapezium is removed. FIG. 13 depicts the dislocation of the joint between the thumb metacarpal and the trapezium with the ligaments and tendons removed to better depict the region where the surgery will occur. FIG. 14 shows the base of the first metacarpal and the trapezial void which exists after excision of the trapezium bone. FIG. 15 shows the void replaced by the prosthesis 51. The insertion of the prosthesis 51 is such that the articulating surface 67 is juxtaposed flush with the articulating surface on the first metacarpal. A ligament 66 is shown being attached at the hole 69 provided in the body of the prosthesis by means of a suture which has been passed through the hole 69 and tied about the ligament 66. Specially roughened or porous surface 71 facilitates the reattachment of the ligament 66. Similarly, a ligament 68 is shown attached at the hole 70 by means of a suture which has been passed through the hole 70 and tied down in the fashion illustrated schematically in FIG. 11A. As can be seen, the design of the implant 51 is such that there is no interference with the natural surrounding members at the joint.

An invention has been provided with several advantages. The prosthesis in accordance with the present invention is relatively easily manufactured employing conventional molding techniques. The prosthesis is surgicallypositioned through relatively simple surgical procedures. The prosthesis has the potential for permitting thumb motion with increased stability, mobility and freedom from pain from that heretofore obtained. Because the device is a total replacement prosthesis for the trapezium, it allows a naturally occurring rotary motion of the thumb joint without luxation or dislocation with respect to the surrounding bones and intercarpal ligamentous structures. Placement of the prosthesis of the invention should result in a successful joint arthroplasty that reestablishes functional joint mechanics. The specially prepared surfaces of the prosthesis as well as the attachment sites for ligaments or tendons help to preserve prosthetic stability. Preservation of these soft tissues contributes substantially to joint stability and function.

While the invention has been shown in only one of its forms, it is not thus limited but is susceptible to various changes and modifications without departing from the spirit thereof.

What is claimed is:

1. A prosthetic device for replacing a trapezium in a human hand, the prosthetic device comprising:
a synthetic body sized and shaped to resemble the removed trapezium, the body having a first articulating surface arranged for engagement with a proximal end of a first metacarpal in the hand through a thumb basilar joint, the device having a second articulating surface arranged for engagement with a distal end of a scaphoid bone of the hand through a scapho trapezio trapezoid joint, the body having a third articulating surface which is arranged for engagement with a trapezoid bone and a second metacarpal bone of the hand, the body also having at least one additional surface which has been formed to promote attachment and adhesion of surrounding ligaments and tissue in the hand after placement of the prosthetic device.

2. The prosthetic device of claim 1, wherein the surface which has been formed to promote attachment and adhesion of surrounding ligaments and tissue in the hand is a porous surface which is provided on a side of the body other than the articulating surfaces.

3. The prosthetic device of claim 1, wherein the surface which has been formed to promote attachment and adhesion of surrounding ligaments and tissue in the hand is a roughened surface which is provided on a side of the body other than the articulating surfaces.

4. The prosthetic device of claim 1, wherein the body is provided with at least one hole which passes wholly or partly through the body to facilitate attachment of surrounding ligaments and tissue of the hand.

5. The prosthetic device of claim 1, wherein the body is provided with a plurality of holes which form attachment sites for attaching sutures, whereby the sutures can be used to attach surrounding ligaments, tendons and other soft tissue to the prosthesis.

6. The prosthetic device of claim 1, wherein the prosthetic body is formed of a non-silicone material.

7. The prosthetic device of claim 1, wherein the prosthetic body is made from a material selected from the group consisting of ceramics, titanium, stainless steel, synthetic diamond and medical grade super alloys based on nickel, cobalt and chromium.

8. A surgical method for treating thumb basil joint arthritis, the method comprising the steps of:
excising the trapezium from the hand of a human;
replacing the excised trapezium with a synthetic body sized and shaped to resemble the removed trapezium, the body having a first articulating surface arranged for engagement with a proximal end of a first metacarpal in the hand through a thumb basilar joint, the device having an oppositely arranged second articulating surface arranged for engagement with a distal end of a scaphoid bone of the hand through a scapho trapezio trapezoid joint, the body having a third articulating surface which is arranged for engagement with a trapezoid bone and a second metacarpal bone of the hand, the body also having at least one additional surface which has been formed to promote attachment and adhesion of surrounding ligaments and tissues in the hand after placement of the prosthetic device.

9. The method of claim 8, wherein the surface which has been formed to promote attachment and adhesion of surrounding ligaments and tissues in the hand is a porous surface which is provided on a side of the body other than the articulating surfaces.

10. The method of claim 8, wherein the surface which has been formed to promote attachment and adhesion of surrounding ligaments in the hand is a roughened surface which is provided on a side of the body other than the articulating surfaces.

11. The method of claim 8, wherein the body is provided with at least one hole which passes wholly or partly through the body to facilitate attachment of surrounding ligaments and tissue of the hand.

12. The method of claim 11, wherein the body is provided with a plurality of holes which form attachment sites for attaching sutures, whereby the sutures can be used to attach surrounding ligaments, tendons and other soft tissue to the prosthesis.

13. The method of claim 1 1, wherein the body is provided with a pair of substantially parallel holes which pass through the body from opposite sides thereof, and wherein a suture is passes through both holes and about a pair of ligaments, one of which is located proximate each of the opposite sides of the body, the suture being tied down with a knot to thereby secure the ligaments to surfaces of the body which have been formed to promote attachment and adhesion of the ligaments to the body.

14. The method of claim 8, wherein the prosthetic body is made from a material selected from the group consisting of ceramics, titanium, stainless steel, synthetic diamond and medical grade super alloys based on nickel, cobalt and chromium.
